(19) United States
(12) Patent Application Publication
(10) Pub. No.: US 2025/0387433 A1
(43) Pub. Date: Dec. 25, 2025

(54) COMPOSITION FOR PREVENTING OR TREATING RETINAL DEGENERATIVE DISEASE CONTAINING EXOSOMES EXTRACTED FROM MESENCHYMAL STEM CELLS

(71) Applicant: Seoul National University Hospital, Seoul (KR)

(72) Inventors: Se Joon Woo, Seongnam-Si (KR); Hye Kyoung Hong, Seongnam-Si (KR); Soo In Seo, Seongnam-Si (KR)

(73) Assignee: Seoul National University Hospital, Seoul (KR)

(21) Appl. No.: 18/842,754

(22) PCT Filed: Feb. 28, 2023
(86) PCT No.: PCT/KR2023/002770

§ 371 Date: May 5, 2025

(30) Foreign Application Priority Data
Mar. 2, 2022 (KR) 10-2022-0026547

(51) Int. Cl. A61K 35/28; A61K 9/00; A61P 27/02

(52) U.S. Cl. CPC A61K 35/28; A61K 9/0019; A61P 27/02

(57) ABSTRACT

One aspect of the present disclosure is a composition for preventing or treating a retinal degenerative disease, capable of preventing or delaying retinal cell degeneration. The composition for preventing or treating a retinal degenerative disease, in one aspect, contains exosomes extracted from human-derived mesenchymal stem cells as an active ingredient.

### CROSS-REFERENCES TO RELATED APPLICATIONS

The present application claims priority to Korean Patent Application No. 10-2022-0026547, filed Mar. 2, 2022, the entire contents of which are hereby incorporated by reference.

### TECHNICAL FIELD

The present invention relates to a composition for preventing or treating a retinal degenerative disease, and more specifically, to a composition for preventing or treating a retinal degenerative disease comprising exosomes extracted from mesenchymal stem cells.

### BACKGROUND ART

A Retinal degeneration disease is a general term for diseases in which pathological changes occur in the retina, which is the nerve tissue of the eye, and the macula, which performs the most important function in the retina, due to genetic abnormalities, aging, inflammation, vascular disease, etc., ultimately leading to blindness. Degeneration of retinal cells is the pathological mechanism of important ophthalmic diseases such as macular degeneration and hereditary retinal diseases. Because retinal cells, like other cells in the nervous system, have poor division and regeneration abilities, there is no effective way to regenerate or replace damaged cells when retinal cells are lost, so there is currently no treatment for retinal cell degeneration. Research into treatment technologies that prevent or delay retinal cell degeneration is urgently needed.

### DETAILED DESCRIPTION OF THE INVENTION

### Technical Problem

The problem to be solved by the present invention is to provide a composition for preventing or treating retinal degenerative diseases, capable of preventing or delaying degeneration of retinal cells.

The problems to be solved by the present invention are not limited to the problems mentioned above, and other problems not mentioned can be clearly understood by those skilled in the art from the description below.

### Technical Solution

According to one embodiment of the present invention to achieve the object, a composition for preventing or treating a retinal degeneration disease may comprise exosomes extracted from human-derived mesenchymal stem cells as an active ingredient.

The mesenchymal stem cells may be tonsil-derived mesenchymal stem cells.

The exosomes may be comprised at a concentration of 1×10¹⁰ to 5×10¹¹ particles/ml.

The exosomes may have a size of 100 to 150 nm.

The retinal degenerative disease may be at least one selected from the group consisting of retinitis pigmentosa (RP), age-related macular degeneration (AMD), diabetic retinopathy, hereditary retinal disease, uveitis, retinal vascular occlusion, optic nerve disease, and glaucoma.

Details of other embodiments are included in the detailed description and drawings.

### Advantageous Effects

In the past, stem cell therapy was attempted by directly injecting stem cells into the eye to treat a retinal degenerative disease, but there were many reports of cases in which the injected stem cells transformed into fibroblasts, resulting in blindness. Therefore, it is difficult to directly apply the stem cell therapy to clinical trials for safety reasons. On the other hand, since the present invention injects into the eye a composition comprising exosomes extracted from tonsil-derived mesenchymal stem cells as an active ingredient, potential risks resulting from stem cell therapy can be avoided. Exosomes included in the composition of the present invention are extracellular vesicles (EVs) that are mediators for delivering secretory factors of stem cells, and have effects such as anti-vascular, anti-inflammatory, anti-fibrotic, immunomodulatory, and neuroprotective. The composition of the present invention has an excellent effect in preventing or treating a retinal degenerative disease by inhibiting the toxicity of substances that cause apoptosis of retina cells.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 is a graph showing the results of a toxicity test on exosomes of the present invention.

FIG. 2 is a graph measuring the cell viability of RPE19 cells by exosomes of the present invention when treated with atRAL.

FIG. 3 is a photograph observed under a microscope before treating the RPE19 cells of FIG. 2 with the WST-1 assay.

FIG. 4 is a graph measuring the inhibitory effect of atRAL toxicity by exosomes of the present invention.

FIG. 5 shows a microscopic image of fluorescently labeled exosomes according to the present embodiment.

FIG. 6 is a graph measuring the thickness of the outer nuclear layer (ONL) and the inner nuclear layer (INL) in the Pde6b gene knockout mouse model according to an embodiment of the present invention.

### DESCRIPTION OF EMBODIMENTS

The advantages and features of the present invention, and the methods for achieving them, will become clearer with reference to the embodiments described in detail below together with the accompanying drawings. However, the present invention is not limited to the embodiments disclosed below, but may be implemented in various different forms, and these embodiments are provided only to make the disclosure of the present invention complete and to fully inform those skilled in the art of the scope of the invention, and the present invention is defined only by the scope of the claims. Like reference numerals refer to like components throughout the specification.

The present invention, in one aspect, provides a composition for preventing or treating a retinal degenerative disease comprising exosomes extracted from human-derived mesenchymal stem cells as an active ingredient.

The present invention, in one aspect, provides a method for preventing or treating a retinal degenerative disease, the method comprising administering an effective amount of culture medium of human-derived mesenchymal stem cells, or exosomes extracted from human-derived mesenchymal stem cells to a subject in need thereof.

The “mesenchymal stem cell” of the present invention refers to an undifferentiated stem cell having the ability to self-replicate and to differentiate into two or more new cells (e.g., bone cells, chondrocytes, muscle cells, fat cells, etc.). The “mesenchymal stem cell” of the present invention may be a human-derived, animal-derived, or plant-derived stem cell, preferably a human-derived mesenchymal stem cell, and more preferably a tonsil-derived mesenchymal stem cell (T-MSC).

The “tonsil-derived mesenchymal stem cell” of the present invention refers to an undifferentiated stem cell having the ability to self-replicate and to differentiate into two or more new cells derived from the tonsil, which is a tissue located inside the throat and in the back of nose which primarily defends our body from substances such as bacteria invading from the outside, and at the same time functions as a lymphoepithelial immune tissue.

The “exosome” of the present invention is an extracellular vesicle (EV) that serves to deliver bio-derived substances such as proteins, fats, metabolites, and nucleic acids to receptor cells.

In the present invention, the “prevention” refers to any act of inhibiting or delaying the onset of a retinal degenerative disease by administering a composition comprising exosomes extracted from tonsil-derived mesenchymal stem cells according to the present invention as an active ingredient. In the present invention, “treatment” refers to all acts of improving or beneficially changing the symptoms of a retinal degeneration disease by administering a composition comprising exosomes extracted from tonsil-derived mesenchymal stem cells according to the present invention as an active ingredient. In the present invention, “improvement” refers to all acts of improving the bad condition of a retinal degeneration disease by administering or ingesting the composition of the present invention to a subject.

In the present invention, the “retinal degenerative disease” refers to a general term for diseases in which pathological changes occur in the retina, which is the nerve tissue of the eye, and the macula, which performs the most important function in the retina, due to genetic abnormalities, aging, inflammation, vascular disease, etc., ultimately leading to blindness. For example, the retinal degenerative diseases include retinitis pigmentosa (RP), age-related macular degeneration (AMD), diabetic retinopathy, hereditary retinal diseases, uveitis, retinal vascular occlusion, optic nerve disease, and glaucoma.

In the composition according to an embodiment of the present invention, exosomes extracted from tonsil-derived mesenchymal stem cells may have an average size (or average diameter) of 100 to 150 nm. In addition, the composition according to one embodiment of the present invention may comprise the exosomes at a concentration of 1×10⁷ to 1×10¹² particles/ml, preferably at a concentration of 1×10¹⁰ to 1×10¹² particles/ml, and more preferably at a concentration of 1×10¹⁰ to 5×10¹¹ particles/ml.

### Embodiment 1. Extraction of Exosomes from Human Tonsil-Derived Mesenchymal Stem Cells (T-MSC)

The conditioned medium used in this embodiment was a conditioned medium for tonsil derived mesenchymal stem cell^{ew}s (T-MSC^{ew}) manufactured by Cellatos Therapeutics Co., Ltd. using human-derived materials approved by the Ewha Medical Center Institutional Review Board (IRB) (EUMC 2019-08-004).

Various methods known in the art can be used to extract exosomes from tonsil-derived mesenchymal stem cells (T-MSCs). For example, stem cells are cultured in a culture medium (DMEM), then subcultured in a serum-free and antibiotic-free medium, the cell culture supernatant is recovered, and the recovered cell culture supernatant is centrifuged and filtered through a 220 nm pore membrane filter to separate and extract exosomes. Specifically, the culture medium was centrifuged at 300×g for 15 minutes and 2000×g for 10 minutes to remove cell debris, and filtration was performed using a 220 nm bottle-top membrane (cell debris removal step). Then, storage agent was removed by flushing with 250 ml of DI water (flushing step). Buffer environment was created inside the device by flushing with 250 ml or more of DPBS (Ca²⁺, Mg²⁺) (Buffer conditioning step). The 250 ml medium was concentrated to a volume of 5 to 10 ml using a 300 kDa MWCO TFF filter (concentration step). At this time, feed pressure was controlled so as not to exceed 0.5 bar. DPBS 7 to 10 times the concentrated volume was added and diafiltration was performed again to a volume of 5 to 10 ml (diafiltration step). At this time, when diluted 7 times or more, small molecule removal of more than 99% was possible. After sample recovery, filtration was performed using a 220 nm bottle-top membrane (product recovery step). Information on the exosomes extracted in this way is as shown in Table 1 below.

TABLE 1

| | | |
|---|---|---|
| Total volume | 5 | ml |
| Mode size | 114.2 ± 7.2 | nm |
| Number concentrations | 2.94 × 10¹¹ ± 2.97 × 10¹⁰ | particles/ml |
| Protein concentrations | 98.31 | mg/ml |

### Embodiment 2. Exosome Toxicity Test (Exosome Toxicity Test In Vitro, RPE19)

RPE19 cells, which are retinal pigment epithelium (RPE) cells, were seeded at 5×10³ cells/well in each 96-well plate and cultured in DMEM/F12 (10% FBS, Penicillin-Streptomycin added) cell culture medium for 24 hours. After the culture, the existing culture medium was removed and replaced with a new cell culture medium, and then exosomes extracted from tonsil-derived mesenchymal stem cells (abbreviated as ‘MSC derived EV’ or ‘EV’) were treated to the wells at various concentrations from 0 or 1×10⁸ to 5×10¹⁰ particles/ml. EZ-cytox cell viability assay reagent (WST-1 cell viability assay kit; DoGenBio, Seoul, Korea) was added 24, 48, and 72 hours after exosome (EV) treatment and incubated in an incubator for 1.5 hours. The optical density (OD) value was measured at 450 nm using a microplate reader.

FIG. 1 is a graph showing the results of a toxicity test on exosomes of the present invention. In FIG. 1, the OD value of the well cultured with RPE19 cells that were not treated with exosomes (EVs) as the control group (None) was set as 100%, and the OD values of the remaining experimental groups were converted based on this. As shown in FIG. 1, in the case of this experimental group, exosomes (EVs) were treated at various concentrations for 24, 48, and 72 hours, but compared to the control group (None), it was confirmed that cell viability was approximately 90% or more, indicating that no cytotoxicity was observed.

### Embodiment 3. Efficacy Test (Efficacy Test In Vitro, RPE19)

RPE19 cells were seeded into 96-well plates at 5×10³ cells/well and cultured in DMEM/F12 (10% FBS, Penicillin-Streptomycin added) cell culture medium for 24 hours. After the culture, the existing culture medium was removed and replaced with a new cell culture medium, and then all-trans-retinal (abbreviated as ‘atRAL’), a toxic substance that induces apoptosis of retina cells, was treated at 25 μM and 50 μM. In addition, exosomes extracted from tonsil-derived mesenchymal stem cells (MSC derived EVs) were treated in well at a concentration of 10 times from 0 or 1×10⁷ to 1×10¹⁰ particles/ml. After 3 hours, EZ-cytox cell viability assay reagent (WST-1 cell viability assay kit; DoGenBio, Seoul, Korea) was added and incubated for 1.5 hours.

The OD values were measured at 450 nm using a microplate reader.

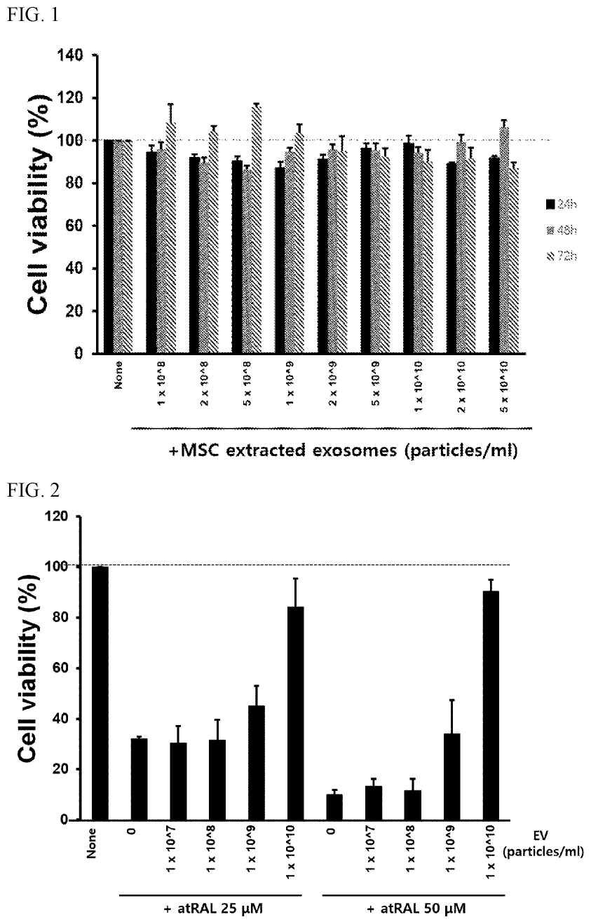

FIG. 2 is a graph measuring the cell viability of RPE19 cells by exosomes of the present invention when treated with atRAL. In FIG. 2, the OD value of the well cultured with RPE19 cells that were not treated with exosomes (EVs) as the control group (None) was set as 100%, and the OD values of the remaining experimental groups were converted based on this. As shown in FIG. 2, the experimental group treated with 25 μM atRAL without adding exosomes (EVs) was confirmed to have approximately 30% cell viability compared to the control group (None), and the experimental group treated with 50 μM atRAL without adding exosomes (EVs) was confirmed to have approximately 10% cell viability compared to the control group (None). On the other hand, when exosomes (EVs) were added and treated, the decrease in cell viability was confirmed to be weakened. In particular, the cell viability of the experimental group treated with 1×10¹⁰ particles/ml of exosomes (EVs) in 25 μM of atRAL was approximately 85%, and the cell viability of the experimental group treated with 1×10¹⁰ particles/ml of exosomes (EVs) in 50 μM of atRAL was approximately 90%. Therefore, through the experiment, it was confirmed that retinal toxicity caused by atRAL was suppressed by exosomes (EV) of the present invention.

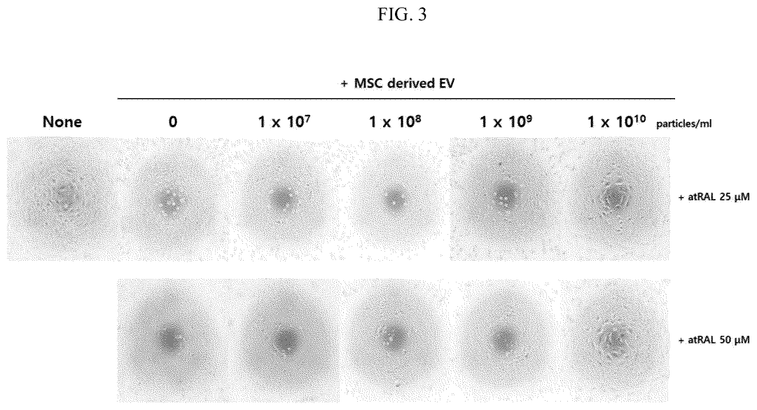

FIG. 3 is a photograph observed under a microscope before treating the RPE19 cells of FIG. 2 with the WST-1 assay. As shown in FIG. 3, in the experimental group treated only with atRAL without exosome (EV) treatment, the number of cells decreased and the shape of the cells became rounder, but when exosomes (EV) were treated together with atRAL at 1×10¹⁰ particles/ml, it was confirmed that there was no significant difference in cell shape compared to the control (None) well.

### Embodiment 4. Efficacy Test (Efficacy Test In Vitro, RPE1)

RPE1 cells were seeded into 96-well plates at 5×10³ cells/well and cultured in DMEM/F12 (10% FBS, Penicillin-Streptomycin added) cell culture medium for 24 hours. After the culture, the existing culture medium was removed and replaced with new cell culture medium containing 50 nM of CytoTox Green dye (Incucyte CytoTox Green, Sartorius, cat #4633) that stains dead cells, and then treated with 50 μM atRAL. In addition, exosomes extracted from tonsil-derived mesenchymal stem cells (MSC derived EVs) were treated in well at a concentration of 10 times from 0 or 1×10⁷ to 1×10¹⁰ particles/ml.

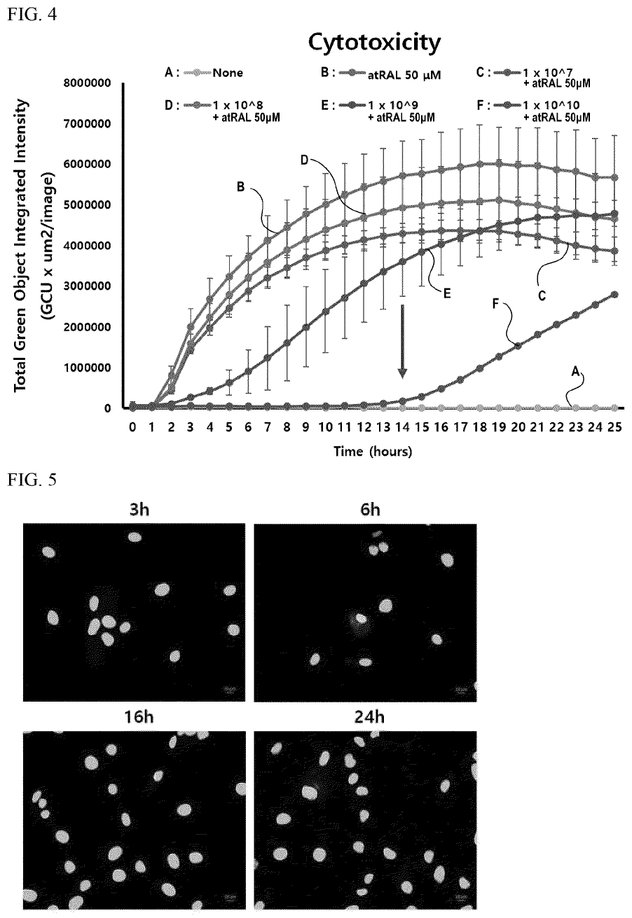

Live cells were observed in bright field every hour using Incucyte live cell imaging equipment (Sartorius), and dead cells were identified by green fluorescence observation for a total of 24 hours. FIG. 4 is a graph measuring the inhibitory effect of atRAL toxicity by exosomes of the present invention, and is the result of measuring the green dye intensity value over time. As shown in FIG. 4, in the experimental group (B) treated with atRAL reagent without adding exosomes (EV), the green dye intensity value steadily increased for about 18 hours due to an increase in apoptotic cells after one hour. In the experimental groups (C, D, E, F) that were additionally treated with exosomes (EV), it was confirmed that the slope of the increase in the green dye intensity value decreased as the concentration of exosomes (EV) increased. In particular, when exosomes (EVs) were treated at 1×10¹⁰ particles/ml, it was confirmed that they were stable for about 14 hours, similar to the control group (None), and that cytotoxicity occurred relatively weakly thereafter.

### Embodiment 5. Localization of Exosomes Labeled with Far-Red (Localization of F(Red)-Tagged EV with RPE1)

RPE1 cells were seeded at 1×10⁴ cells/well in each 8-well chamber slide and cultured in DMEM/F12 (10% FBS, Penicillin-Streptomycin added) cell culture medium for 24 hours. After the culture, the existing culture medium was removed and replaced with new cell culture medium, and then exosomes extracted from tonsil-derived mesenchymal stem cells (MSC derived EVs) labeled with far-red fluorescence were treated at 5×10⁹ particles/ml. After 3, 6, 16, and 24 hours, the culture medium was removed, the cells were fixed using 4% paraformaldehyde (PFA), and then observed under a fluorescence microscope after DAPI staining. FIG. 5 shows a microscopic image of fluorescently labeled exosomes according to the present embodiment. As shown in FIG. 5, it was confirmed that exosomes (EVs) were present within the cells even 24 hours after exosome (EV) treatment. That is, it was confirmed through this embodiment that exosomes directly move into the RPE1 cytoplasm and exert a therapeutic effect.

### Embodiment 6. Pde6b Gene Knockout Rat Model (In Vivo Pde6b Knockout Rat)

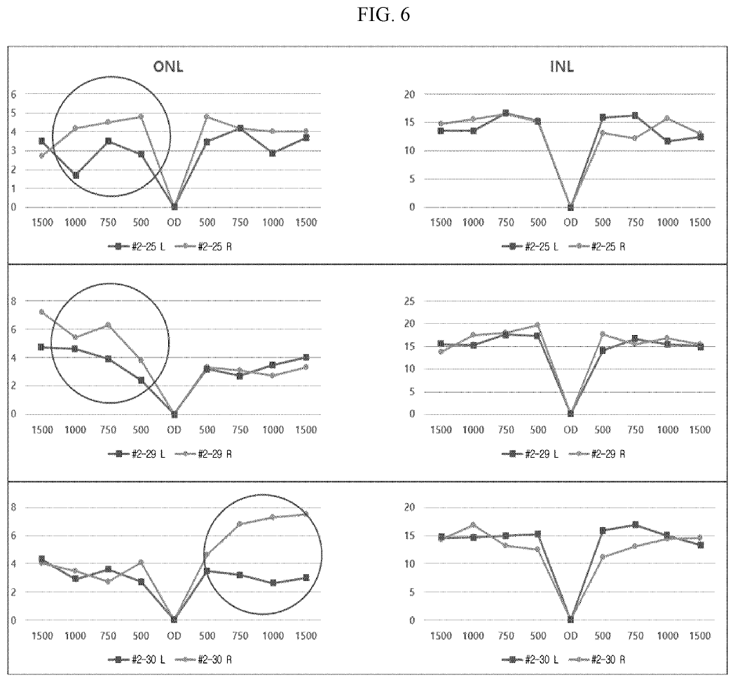

Based on the previous in vitro embodiment, the efficacy of exosomes extracted from tonsil-derived mesenchymal stem cells was confirmed in vivo using PDE6b knockout rats. After anesthetizing PDE6b knockout rats, 3×10¹¹ exosome (EV) particles/ml were injected intravitreously, and fundus photography was performed. The concentration of exosomes (EVs) in rat vitreous body (vitreous volume: 13-20 μl) is 1.5×10⁷ to 2.3×10⁷ particles/μl. Rat eye balls were injected with exosomes (EVs) and removed 15 days later to create paraffin blocks. Paraffin blocks of rat eyes were sectioned at 4 μm thickness to create slides, and H&E staining was performed. Using a slide scanner, the thicknesses of the outer nuclear layer (ONL) and inner nuclear layer (INL) of the retina were measured at 500, 750, 1,000, and 1,500 m to the left and right of the optic disc. FIG. 6 is a graph measuring the thickness of the outer nuclear layer (ONL) and the inner nuclear layer (INL) in the Pde6b gene knockout mouse model according to an embodiment of the present invention. To maintain objectivity during thickness measurement, the sample number was covered and the measurement was performed blindly, and each area was measured three times to derive the average value. The left eye, which was not injected with exosomes (EV), was used as a control group (#2-25 L, #2-29 L, #2-30 L), and only the right eye, which was used as an experimental group (#2-25R, #2-29R, #2-30R), was injected with exosomes (EV). As shown in FIG. 6, the thickness of the outer nuclear layer (ONL) of the right eye injected with exosomes (EV) was confirmed to increase compared to the left eye, which is the control group. The increase in the thickness of the outer nuclear layer (ONL) on only one side of the optic disc in the experimental group was because exosomes (EVs) were injected only in that direction.

Although the embodiments of the present invention have been described with reference to the attached drawings, those skilled in the art will understand that the present invention can be implemented in other specific forms without changing the technical idea or essential features thereof. Therefore, it should be understood that the embodiments described above are exemplary in all respects and not restrictive.

1. A method for preventing or treating a retinal degenerative disease, the method comprising administering an effective amount of culture medium of human-derived mesenchymal stem cells, or exosomes extracted from human-derived mesenchymal stem cells to a subject in need thereof.

2. The method for preventing or treating a retinal degenerative disease of claim 1, wherein the mesenchymal stem cells are tonsil-derived mesenchymal stem cells.

3. The method for preventing or treating a retinal degenerative disease of claim 1, wherein the exosomes are administered at a concentration of 1×10¹⁰ to 5×10¹¹ particles/ml.

4. The method for preventing or treating a retinal degenerative disease of claim 1, wherein the exosomes have a size of 100 to 150 nm.

5. The method for preventing or treating a retinal degenerative disease of claim 1, wherein the retinal degenerative disease is at least one selected from the group consisting of retinitis pigmentosa (RP), age-related macular degeneration (AMD), diabetic retinopathy, hereditary retinal disease, uveitis, retinal vascular occlusion, optic nerve disease, and glaucoma.

6. The method for preventing or treating a retinal degenerative disease of claim 1, wherein the administration is an intraocular injection.

7. The method for preventing or treating a retinal degenerative disease of claim 1, wherein the subject needs to suppress the toxicity of a substance that induces apoptosis of retina cells.

8. The method for preventing or treating a retinal degenerative disease of claim 7, wherein the apoptosis is induced by atRAL.
